(19) United States
(12) Patent Application Publication
(10) Pub. No.: US 2021/0267514 A1
(43) Pub. Date: Sep. 2, 2021

(54) METHOD AND APPARATUS FOR MONITORING EMOTIONAL COMPATIBILITY IN ONLINE DATING

(71) Applicant: Q-Tec Systems LLC, Scottsdale, AZ (US)

(72) Inventor: Roger J. Quy, Scottsdale, AZ (US)

(21) Appl. No.: 17/182,302

(22) Filed: Feb. 23, 2021

Related U.S. Application Data
(63) Continuation of application No. 14/251,774, filed on Apr. 14, 2014, now abandoned.
Application No. 14/251,774 is a continuation-in-part of application No. 13/151,711, filed on Jun. 2, 2011, now Pat. No. 8,700,009.
(60) Provisional application No. 61/350,651, filed on Jun. 2, 2010.

(51) Int. Cl. A61B 5/16; H04W 4/21; A61B 5/369

(52) U.S. Cl. CPC A61B 5/165; H04L 67/12; A61B 5/369; H04W 4/21

(57) ABSTRACT

Methods, devices, and systems provide for capturing and sharing objective emotion data in dating interactions for the selection of suitable partners, or to enhance social dynamics in online interactions. An emotion monitoring device (EMD) measures physiological signals, obtained from biosensors, captured from a couple during a face-to-face or online dating interaction, and computes emotion data. The emotion data for each person is transmitted to an internet server, and each person shares their emotion data with the other during the interaction. The emotion data are displayed to each person on a virtual or augmented reality device. In an alternative embodiment for virtual dating games or other online social interactions, the emotion data of a group of users, each equipped with an EMD, are captured, shared and displayed.

### CROSS-REFERENCE TO RELATED APPLICATIONS

This application is a continuation of U.S. application Ser. No. 14/251,744, filed Apr. 14, 2014, which is a continuation-in-part of U.S. patent application Ser. No. 13/151,711, filed: Jun. 2, 2011, now U.S. Pat. No. 8,700,009, which claims priority to U.S. Provisional Patent Application Ser. No. 61/350,651, filed Jun. 2, 2010, entitled “METHOD AND APPARATUS FOR INTERACTIVE MONITORING OF EMOTION”, the entirety of each being incorporated by reference herein.

### FIELD OF THE INVENTION

The present invention relates to monitoring the emotions of persons using biosensors and the use of such monitoring data.

### BACKGROUND OF THE INVENTION

It is known that human emotional states have underlying physiological correlates reflecting activity of the autonomic nervous system. A variety of physiological signals have been used to detect emotional states. However, it is not easy to use physiological data to monitor emotions accurately because physiological signals are susceptible to artifact, particularly with mobile users, and the relationship between physiological measures and positive or negative emotional states is complex.

A standard model separates emotional states into two axes: arousal (e.g., calm-excited) and valence (negative-positive]. Thus emotions can be broadly categorized into high arousal states, such as fear/anger/frustration (negative valence) and joy/excitement/elation (positive valence); or low arousal states, such as depressed/sad/bored (negative valence) and relaxed/peaceful/blissful (positive valence). An increasing number of people are turning to the internet to find partners for dating or marriage. According to a 2013 study by the Pew Research Center, 10% of Americans have used an online dating site (or mobile App); 46% of these users said finding someone for a long-term relationship is a major reason that they use these sites. However, online dating services often disappoint in the selection of candidates provided to the users. One reason for the poor compatibility of prospective partners is because the services frequently rely on profiles prepared by the users to describe themselves, and these profiles are often inaccurate if not deliberately misrepresentative. The 2013 Pew study found that 54% of users reported someone else had seriously misrepresented themselves in an online dating profile. Furthermore, the Federal Trade Commission has warned users of online dating services to be aware of scammers who create fake profiles to build online relationships with their victims.

In order to improve on self-published profiles, some dating services employ surveys to match couples. However, the information gathered by these surveys tends to be superficial. Others utilize a psychographic questionnaire to develop a character profile. A proprietary questionnaire has been developed to approximate the satisfaction a person has in relationships with others and identify candidates so as to reduce matches between people who are likely to have conflicting relationships (U.S. Pat. No. 6,735,568). A problem with questionnaires in general, however, is that users naturally try to paint themselves in the best possible light. Moreover, emotional compatibility is not easily addressed by questionnaires, because many people are aware of, or are unwilling to be honest about, their emotional makeup.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY OF THE INVENTION

Systems and methods according to present principles provide ways to monitor emotional states to determine the emotional compatibility of couples in dating or matchmaking. By monitoring the physiological correlates of emotional states, an objective emotional profile can be obtained. Furthermore, the physiologic data are harder to fake than responses to a questionnaire because the autonomic nervous system acts at a sub-conscious level. One exemplary object of certain implementations of the present invention is to monitor emotional states corresponding to various standardized conditions, or in face-to-face interactions, so as to provide a more revealing and realistic means to select compatible partners for dating or marriage.

One or more implementations of this invention overcome certain of the disadvantages of the prior art by incorporating a number of features to reduce artifact and improve the detection and monitoring of emotional states. In implementations, an emotion recognition algorithm derives emotion arousal and valence indices from physiological signals. These emotion-related data are calculated from physiological signals and communicated to and from a software application. In one implementation to select emotionally compatible couples, the emotion data are captured from a pool of users in response to standardized stimuli and processed to determine each user's emotion profile. A software algorithm is then used to categorize and match the profiles according to empirically derived or otherwise set criteria. In another implementation, the emotion data monitored from a couple are shared in the course of an online, or face-to-face, dating interaction (e.g., during speed-dating). In an implementation for a dating social game, the emotion data of multiple players are monitored in a virtual, or face-to face (e.g., a party), context and shared for display to the players and others.

Previous systems to detect emotions have typically been designed for laboratory use and are based on a computer. In contrast, this system is designed for personal use and can be based on a smart mobile device, e.g., iPhone®, thus enabling emotions to be monitored in everyday surroundings and casual settings. Moreover, the system is designed for multiple users that can be connected in an interactive network whereby emotion data can be collected and shared. The sharing of emotion data, made possible by cellular communications, can be a way to enrich the experiences of users interacting with a variety of social communities, media and, entertainment.

People are often not aware of transient emotional changes so monitoring emotional states can enrich experiences for individuals or groups. Other applications of emotion monitoring include entertainment, such as using emotion data for interactive gaming, or interactive television and movies. Another application is for personal training—for example, learning to control emotions and maintain a healthy mental attitude for stress management, yoga, meditation, sports peak performance and lifestyle or clinical management. Others have used physiological signals such as heart rate and skin conductance (also known as galvanic skin response or GSR), for biofeedback training or to control games and other software. In implementations according to present principles, the physiological data are processed to obtain metrics for emotional arousal level and/or valence that can provide data useful in dating or matchmaking, as well as to provide control signals for feedback and interactivity.

Multiple users equipped with emotion monitors can be connected directly, in peer-to-peer networks or via the internet, with shared emotion data. Applications include multiplayer games, online dating services, team sports, or other group activities. With many users connected in a network, emotion data can enhance social games, media, and communities. The emotion data can be captured and analyzed for marketing purposes. Emotion ratings can be collected via the internet based on user responses to a variety of media, including written content, graphics, photographs, video and music. Emotional reactions to other sensory input such as taste and olfactory tests could also be obtained. The media used to engender emotion data can be standardized to provide a consistent experience, e.g., for users of an online dating service.

In more detail, implementations provide systems and methods for interactive monitoring of emotion by recording one or more physiological signals, in some cases using simultaneous measurements, and processing these signals with a novel emotion detection algorithm, providing a display of emotion data, and using the data to interact with other users, games or software. The emotion data can be transmitted to an internet server and shared by more than one user to form an emotion network for multiplayer, interactive games and social communities.

Biosensors record physiological signals that relate to changes in emotional states, such as skin conductance, skin temperature, respiration, heart rate, blood volume pulse, blood oxygenation, electrocardiogram (ECG), electromyogram (EMG), and electroencephalogram (EEG). For a variety of these signals, either wet or dry electrodes are utilized. Alternatively, photoplethysmography (PPG), which utilizes a light source and light sensor, can be employed, e.g., to record heart pulse rate and blood volume pulse. The biosensors can be deployed in a variety of forms, including a finger pad, finger cuff, ring, glove, ear clip, wrist-band, chest-band, or head-band. The sensors can be integrated into the casing of a mobile game console or controller, a TV remote, a computer mouse, or other hand-held device; or into a cover that fits onto a hand-held device, e.g., a mobile phone. In some cases, the biosensors may be integrated into an ancillary game controller that is in turn in signal communication with a standard game controller. In other cases, the biosensors may be integrated into a virtual reality headset, e.g. for affective computing.

In some implementations, a plurality of biosensors may simultaneously record physiological signals, and the emotion algorithm may receive these plurality of signals and employ the same in displaying emotion data in responding to the emotion data, such as for an emotion network or for the control of interactive games. In such cases, a plurality of biosensors may be employed to detect and employ emotion signals in the game. Physiological signals are easily contaminated by noise from a variety of sources, especially movement artifacts. A variety of methods are used to improve the signal to noise ratio and remove artifact. Electrical biosensors include electromagnetic shielding, e.g., a Faraday cage, to reduce environmental noise. Since the contact between the biosensor and underlying skin could be poor (e.g., through clothing or hair), the signals may be coupled to a very high-impedance input amplifier. Capacitive-coupled biosensors can be used in some applications. Another strategy is to use an array of biosensors in the place of one, which allows for different contact points or those with the strongest signal source to be selected, and others used for artifact detection and active noise cancellation. An accelerometer can be attached to the biosensor to aid monitoring and cancellation of movement artifacts.

The signal may be further processed to enhance signal detection and remove artifacts using algorithms based on blind signal separation methods and state of the art machine learning techniques. By way of illustration, when detecting beat-to-beat heart rate from a biosensor designed for everyday use by consumers (in contrast to the medical sensors typically used in a clinical or research setting) the heart QRS complexes are identified via a hybrid pattern recognition and filter-bank method with dynamic thresholding. Heart beats thus detected are then fed to a probabilistic (Bayesian) tracking algorithm based on Gauss-Hermite, Kalman filtering, thereby increasing robustness to noise and insensitivity to ECG arrhythmia while maintaining responsiveness to rapidly changing heart rates. Such signal processing may be particularly useful in cleaning data measured by such biosensors, as user movement can be a significant source of noise and artifacts.

The physiological signals are transmitted to an emotion monitoring device (EMD) either by a direct, wired connection or wireless connection. Short range wireless transmission schemes may be employed, such as a variety of 802.11 protocols (e.g., Wi-Fi), 802.15 protocols (e.g., Bluetooth® and Zigbee™), other RF protocols, (e.g., ANT), telecommunication schemes (e.g., 3G, 4G) or optical (e.g., infra-red) methods. The EMD can be implemented on a number of devices, such as a mobile phone, game console, netbook computer, tablet computer, laptop, personal computer, or proprietary hardware such as a virtual reality headset. The EMD can be a wearable device, e.g., smart watch, eyewear, or apparel. The EMD processes the physiological signals to derive and display emotion data, such as arousal and valence components. Others have used a variety of apparatus and methods to monitor emotion, typically some measure reflecting activation of the sympathetic nervous system, such as indicated by changes in skin temperature, skin conductance, respiration, heart rate variability, blood volume pulse, or EEG. Deriving emotion valence (e.g., distinguishing between different states of positive and negative emotional arousal) is more complex. Some alternative approaches that can be employed to distinguish between emotional states include the analysis of body heat signatures or facial micro-expressions, (e.g., as monitored by cameras, especially those head mounted such as on eyewear, or by recording EMG signals).

Implementations of the invention may employ algorithms to provide a map of both emotional arousal and valence states from physiological data. In one example of an algorithm for deriving emotional states, the arousal and valence components of emotion are calculated from measured changes in skin conductance level (SCL) and changes in heart rate (HR), in particular the beat-to-beat heart rate variability (HRV). Traditionally, valence was thought to be associated with HRV, in particular the ratio of low frequency to high frequency (LF/HF) heart rate activity. By combining the standard LF/HF analysis with an analysis of the absolute range of the HR (max-min over the last few seconds), emotional states can be more accurately detected. By way of illustration, one algorithm is as follows: If LF/HF is low (calibrated for that user) and/or the heart rate range is low (calibrated for that user) this indicates a negative emotional state. If either measurement is high, while the other measurement is in a medium or a high range, this indicates a positive state. A special case is when arousal is low; in this case LF/HF can be low, while if the HR range is high, this still indicates a positive emotional state. The accuracy of the valence algorithm is dependent on detecting and removing artifact to produce a consistent and clean HR signal.

A method of SCL analysis is also employed for deriving emotional arousal. A drop in SCL generally corresponds to a decrease in arousal, but a sharp drop following a spike indicates high, not low, arousal. A momentary SCL spike can indicate a moderately high arousal, but a true high arousal state is a series of spikes, followed by drops. Traditionally this might be seen as an increase, then decrease, in arousal, but should instead be seen as a constantly high arousal. Indicated arousal level should increase during a series of spikes and drops, such that the most aroused state, such as by anger if in negative valence, requires a sustained increase, or repeated series of increases and decreases, in a short period of time, not just a single large increase, no matter the magnitude of the increase. The algorithm can be adapted to utilize BVP as the physiological signal of arousal.

Analysis of EEG data is also utilized to derive emotion states. Asymmetries, phase synchronization, and coherences of different regions and frequency bands (e.g., alpha, theta, beta, and gamma) or event-related potentials (ERPs) and even-related synchronization (ERS) may provide correlates of emotion. Reduced spectral power in the alpha band over the left frontal region relative to the right frontal region corresponds to increased cortical activation and has been shown to reflect an approach verses avoidance motivation. However, frontal alpha asymmetry alone is not a reliable indicator of emotional valence because anger as well as positive emotions can engender an approach response. Hence an algorithm evaluates EEG data together with other indicators of arousal and valence (e.g., HRV and SCL indicators) to provide a consistent measure of emotion states. Nevertheless, the approach/avoidance responses indicated by frontal asymmetry of alpha power in the EEG data may be utilized in selecting compatible partners for dating and matchmaking, or selecting individuals who may work well together, e.g., for recruiting members to a team, workplace, or organization.

The above-described emotion-deriving algorithms are believed to have certain advantages in certain implementations of the invention. However, other ways of deriving emotion variables may also be employed. As may be seen above, these algorithms generally derive emotion data, which may include deriving values for individual variables such as level of stress. However, they also can generally derive a number of other emotion variables, and as such may be thought of as occupying an abstraction layer above, e.g., devices that propose to calculate a single variable such as stress from measurements of skin conductance or heart rate variability. The emotion-deriving algorithms may be implemented in a software application running in the EMD, or in firmware, e.g., a programmable logic array, read-only memory chips, or other known methods, or running on an internet server.

The system is designed to calibrate automatically each time it is used; also baseline data are stored for each user, so the algorithm improves automatically as it learns more about each user's physiological profile. Accuracy of emotion detection is improved with the addition of more physiological data—such as skin temperature, respiration, or EEG.

The emotional arousal and valence data can be expressed in the form of a matrix displaying emotional states. The quadrants in the matrix can be labeled to identify different emotional states depending on the algorithm, e.g., feeling “angry/anxious, happy/excited, sad/bored, relaxed/peaceful”. The data can be further processed to rotate the axes, or to select data subsets, vectors, and other indices such as “approve/disprove”, “like/dislike”, “agree/disagree”, “feel good/feel bad”, “approach/avoidance”, “good mood/bad mood”, “calm/stressed”; or to identify specific emotional states, such as being “centered” or “in the zone” (e.g., for sports peak performance). The emotional states and scores can be validated against standard emotional stimuli (e.g., the International Affective Picture System). In addition with large data sets, techniques such as machine learning, data mining, or statistical analysis can be used to refine the analysis and obtain specific emotional response rating scales. Statistical tools (e.g., discriminant and variance analysis) can be employed to categorize a user's emotional responses to a variety of stimuli so as to provide a comprehensive emotion matrix or profile of the user. The emotion profiles can be sorted and categorized according to external data, e.g., empirical criteria, quantifying the success of dating or longer-term relationships, as measured between individuals with comparisons of their derived emotion profiles for compatibility. Other implementations may be seen, e.g., for recruiting members to a team, workplace, or organization; or for enhancing the social dynamics of participants in group activities, negotiations, business discussions, and the like.

It can be helpful for emotion data to be displayed to the user in graphical form. Other visual or auditory feedback can be utilized, such as a color code or symbol (e.g., “emoticon”) representing the emotional states. The emotion data optionally may then be transmitted to an internet server, or a cloud infrastructure, via a wired or wireless telecommunication network. An internet server may send a response back to the user; and with multiple users, the emotion data of one user may be transmitted from the server to be displayed on the EMD of other users. The server application program stores the emotion data and interacts with the users, sharing emotion data among multiple users as required. The emotion data may be incorporated in local, multiplayer, and social games or online communities that have been designed or adapted to interact with a user's emotional response, so that characters, events, objects or other players can respond to a player's emotions. Additionally, emotion data may be obtained, transmitted, analyzed, and displayed in response to online content that is downloaded to the EMD. The emotion rating scores may be statistically manipulated, analyzed, or made available to social communities and online search engines, as required.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1 illustrates a general embodiment of an emotion monitoring network according to present principles to determine emotional compatibility of users of an online dating service.

FIG. 2 illustrates two emotion monitoring devices sharing the emotion data of a couple during a dating interaction.

FIG. 3 illustrates a network of mobile emotion monitoring devices utilized for a multiplayer, dating game.

FIG. 4 illustrates an embodiment of an emotion monitoring device based on a mobile phone wirelessly connected to biosensors.

FIG. 5 illustrates an embodiment of an emotion monitoring device based on biosensors integrated into the casing of a mobile phone.

FIG. 6 illustrates an embodiment of an emotion monitoring device based on biosensors integrated into a virtual reality headset.

FIG. 7 illustrates a flowchart of a general method for operating an emotion monitoring network.

FIG. 8 illustrates a flowchart of a method for an emotion monitoring network to determine emotional compatibility of users of an online dating service.

Like reference numerals refer to like elements throughout. Elements are not to scale unless otherwise indicated.

### DETAILED DESCRIPTION OF THE PREFERRED EMBODIMENTS

Various acronyms are used for clarity herein. Definitions are given below.

The term “subject” as used herein indicates a human subject. The term “user” is generally used to refer to the user of the device, which may be synonymous with the subject. The term “signal communication” is used to mean any type of connection between components that allows information to be passed from one component to another. This term may be used in a similar fashion as “coupled”, “connected”, “information communication”, “data communication”, etc. The following are examples of signal communication schemes. As for wired techniques, a standard bus, serial or parallel cable may be used if the input/output ports are compatible and an optional adaptor may be employed if they are not. As for wireless techniques, radio frequency (RF) or microwaves, and optical techniques, including lasers or infrared (IR), and other such techniques may be used. A variety of methods and protocols may be employed for short-range, wireless communication including IEEE 802 family protocols, such as Bluetooth® (also known as 802.15), Wifi (802.11), ZigBee™, Wireless USB and other personal area network (PAN) methods, including those being developed. For wide-area wireless telecommunication, a variety of cellular, radio satellite, optical, or microwave methods may be employed, and a variety of protocols, including IEEE 802 family protocols (e.g., 802.11, 802.16, or 802.20), Wi-Fi, WiMax, UWB, Voice over IP (VOIP), Long-Term Evolution (LTE), and other wide-area network or broadband transmission methods and communication standards being developed. It is understood that the above list is not exhaustive.

Various embodiments of the invention are now described in more detail.

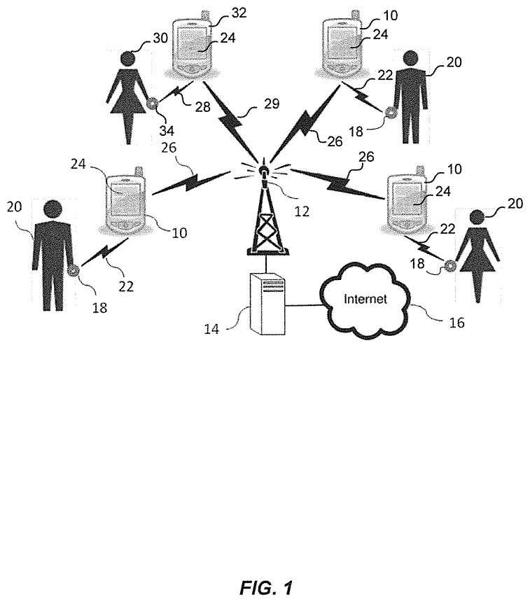

Referring to FIG. 1, a system according to present principles is shown for monitoring emotion data from one or more subjects connected in a network. A subject 20 is in contact with one or more biosensors 18 to record physiological signals. The biosensors can be deployed in a variety of forms, including a finger pad, finger cuff, ring, glove, ear clip, wrist-band, chest-band, head-band, or headset. Other varieties of biosensors will also be understood; for example, the biosensors may be embodied in eyewear, and may monitor facial expressions or micro-expressions, eye movements, or the like. The physiological signals are transmitted to an emotion monitoring device (EMD) 10, such as a mobile device, e.g., a smart phone, by a wired or short-range wireless connection 22. As described above, EMD 10 further processes the physiological signals and an algorithm derives emotion data from the signals, such as arousal and valence indices. Screen 24 displays emotion data to subject 18.

EMD 10 is connected to a telecommunication network 12 via a wide area, wired or wireless connection 26. The telecommunication network 12 is connected to server 14 that is part of the internet infrastructure 16. EMD 10 optionally transmits the emotion data to a website associated with an application program running on computer readable media (CRM) in server 14, which receives, processes and responds to the data. The computer readable media in server 14 and elsewhere may be in non-transitory form. A response can be transmitted back to EMD 10. The server may also transmit emotion data via connection 28 to be displayed to a remote subject 30. The remote subject 30 is equipped with an EMD 32 and may also have biosensors 34 and may similarly transmit emotion data via connections 29, 28 to the internet server 14. (One remote subject is illustrated, but a plurality is similarly equipped.) The server application program stores the emotion data and interacts with the subjects, including sharing emotion data among the network of users.

Emotion data may be derived from the signals either using an algorithm operating on the EMD 10 or using an algorithm operating on the server 14, or the two devices may work together to derive emotion data, such as arousal and valence indices.

The system of FIG. 1 may be employed to match users of an online dating service, based on their emotional responses to standardized stimuli. A plurality of subjects 20, 30 is each in contact with one or more biosensors 18, 34, respectively, to record physiological signals, which are transmitted by a wired or wireless connections 22, 29 to EMDs 10, 32. A display screen 24, which may be incorporated in the EMD or a separate device (e.g., a desktop computer), displays a series of stimuli to each subject. The stimuli are downloaded to the display from a server 14 connected to the internet 16 and can include a variety of written content, graphics, photographs, video, audio, or music. The EMD derives emotion data corresponding to the stimuli from the physiological signals, and transmits the emotion data to the internet server 14, via wired or wireless connections 26, 28 in a communications network 12, as described above.

A software application running on the internet server 14 calculates an emotion matrix or profile for each subject based on their emotional arousal and valence responses to each stimulus. The application further uses an algorithm to sort and categorize the emotion profiles. Then the probability of compatibility between pairs is calculated utilizing measures of the success of relationships from other variables and data sources. For example, the emotion profiles of couples who are happily married can be collected and compared with those who underwent divorce. The algorithm can employ techniques such as determinant or variance analysis, case-based reasoning, rules-based systems, neural networks, machine learning, or other such analysis techniques as are known.

It will be understood that each of the stimuli, emotion data, application program, algorithm, external data source, or other analysis techniques may physically reside on more than one server or different servers (e.g., on a cloud of servers) for storage or multiple processing purposes.

The stimuli used to determine emotional compatibility of prospective partners are chosen to reflect issues important to the success of dating or marriage, e.g., photographs of children and babies, scenes illustrating different attitudes about money, or about sex. Videos of actors portraying couples in various scenarios can be used to explore deeper compatibility issues that are much too complex for questionnaires. A consistent set of stimuli is used to provide standardized metrics across subjects. The set is updated as measures of the success of emotion compatibility matching for relationships are obtained, using analytical techniques such as statistical methods, machine learning, and the like.

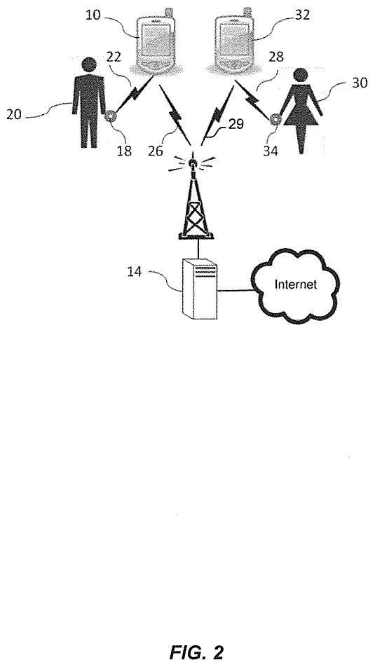

Referring to FIG. 2, an implementation according to present principles is illustrated to monitor and share emotion data from a couple during a face-to-face or online dating interaction. One subject member 20 of the couple is in contact with one or more biosensors 18 to record physiological signals, which are transmitted to an EMD 10 by a wired or short-range wireless connection 22. The EMD derives emotion data and transmits it via a wired or wireless connection 26, e.g., using a mobile network, to an internet server 14 as described above. The server transmits the emotion data via a similar connection 28 to be displayed on the EMD 32 of the other subject member 30 of the couple. The EMD 32 of the other subject member 30 similarly derives emotion data from biosensors 34 and transmits the data via connection 28, to the internet server 14, which in turn transmits it to be displayed on the EMD 10 of the first subject member 20. Thus the couple can see each other's emotional responses as they converse, which will provide them with insightful information about their compatibility. The emotion data for the interaction can be stored on internet server 14 for later review, which may be advantageous in scenarios such as speed-dating to enable users to compare the compatibility of a series of prospective partners. In some implementations, emotion data is provided to another member for review, as shown; in other implementations, the emotion data of multiple individuals is received and compared to determine likely matches.

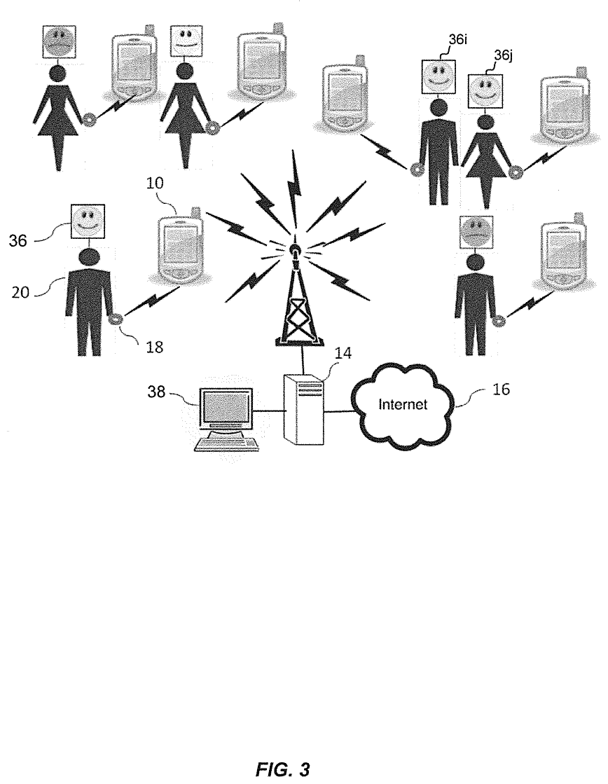

Referring to FIG. 3, an emotion monitoring network is illustrated where emotion data are shared in a dating social game. The game players may be located at the same place (e.g., at a party or other social event) or remotely located for an online game. Each player 20 is in contact with biosensors 18 to record physiological signals and derive emotion data from the signal using an EMD 10 as described above. The EMD of each player transmits the emotion data to the internet 16, where it can be shared with other players, as also described above. In addition, for a group dating or party game, each player wears a device 36 that depicts the current emotional state of the player. In one embodiment, this device is a color-coded light or an emoticon display worn on the body, e.g., mounted on an EEG headset that is used to derive emotion data. (One player is illustrated but each is similarly equipped.) In an online or virtual reality environment, the players may be represented by avatars wherein the emotion data of the players are reflected by the avatars, e.g., by the emoticons, or via facial expressions, colors, symbols, auras, or the like. The emotion data from all the players may be aggregated by an internet server 14 and broadcast to the group on display screen 38 to show the communal mood or emotional “temperature” of the party; and transmitted via social media to identify “social hotspots” in the community.

The implementation illustrated in FIG. 3. may be adapted for monitoring and sharing emotion data in other group activities, e.g., monitoring the collective mood and emotion data of participants in business meetings, video conferences, or negotiations.

Details of specific hardware and software which may be employed to implement the above principles is now described.

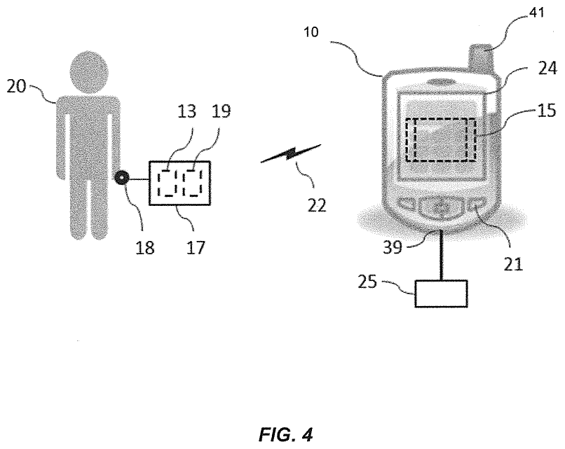

Referring to FIG. 4, an embodiment of EMD 10 is shown based on a smart, mobile phone 11. One or more biosensors 18 measure physiological signals from a subject 20. A variety of types of biosensors may be employed that measure signals related to changes in emotional states, such as skin conductance, skin temperature, heart rate, blood volume, pulse, blood oxygenation, ECG, and EEG. For a variety of these signals, either wet or dry electrodes, or alternatively, PPG optical sensors, can be employed. Implantable sensors may also be utilized. The biosensors may be incorporated in a finger pad, finger ring, ear clip (e.g., attached to a phone earpiece), wrist-band, chest-band, head-band, hat, or adhesive patch as a means of attaching the biosensors to the subject. The biosensors may also be implemented within eyewear or virtual reality headset.

The signals are amplified and processed to reduce artifact in a signal processing unit (SPU) 17. An accelerometer 13 optionally may be included to aid monitoring and cancellation of movement artifacts. A short-range wireless transmitter 19 is employed to transmit the signals via connection 22 (e.g., Bluetooth®) to a web-enabled, mobile phone 11 (e.g., iPhone® or Android®). An optional adapter 25 connected to the generic input/output port or “dock connector” 39 of the mobile device may be employed to receive the signals in some implementations, or even to perform the measurements. Alternatively, SPU 17 can connect by means of a direct or wired connection to the mobile phone. An application program 15 is downloaded from an internet server to a computer readable medium in the mobile phone. The application program receives and processes the physiological signals and includes an algorithm to derive emotion data. Alternatively, the algorithm may be operated on a server to which the mobile device is in data communication. The application program includes a user interface to display the emotion data on screen 24, and for the subject to manually enter information by means of a keyboard, buttons or touch screen 21. As noted in FIG. 1, the mobile device may optionally transmit the emotion data via wireless connection 26 to the internet server, and may receive emotion data of other users, these transmissions either through the internet server or peer-to-peer.

It will be clear to one of ordinary skill in the art given this teaching that mobile device may be any type of wireless device such as a mobile phone, tablet computer, desktop computer, laptop computer, PC, game controller, TV remote controller, computer mouse, or other hand-held device, or a wearable device, such as a smart watch, provided that such devices have equivalent functionality. The advantage of a web-enabled wireless phone (in contrast to a personal computer or video game console) is that it enables a user's emotions to be monitored and shared with others when the user is fully mobile in a wide-area environment, such as walking around a store. However, the limited amount of memory, processing capability, and display size available on a mobile phone in comparison to a computer (PC) constrains the functionality of the software running on the phone. Application program 15 is thus designed to suit the functional constraints of mobile phone 11. In the case of an emotion network that might encompass a large number of users, it is important that the internet infrastructure is employed for significant application processing and storage of emotion data so that less memory and processing capabilities become necessary on the mobile phone, thus freeing memory and processing for receiving physiological signals and in many cases for calculating the related emotion data.

The advent of web-enabled mobile phones has brought increased functionality for sending and receiving data from the internet. A web-enabled or smart phone (e.g., iPhone®) is distinguished from conventional cellular phones by features such as a web browser to access and display information from internet web sites. In addition, modern, web-enabled mobile phones run complete operating system software that provides a platform for mobile application programs or “apps”. Third party applications, such as described here, can be downloaded immediately to the phone from a digital distribution system website (e.g., iTunes®) over a wireless network without using a PC to load the program. With increased functionality, the smart phone operating systems can run and multitask applications that are native to the underlying hardware, such as receiving data from an input port and from the internet, at the same time as running other applications using the data. Similarly, a web-enabled tablet (e.g., iPad®) has the advantage of enhanced mobility, by reason of compactness, in contrast to a conventional desktop or even laptop computer; and it has the advantages of an operating system that can run a web browser, download apps from a web site, and multitask application programs, e.g., simultaneously receiving data and running a program to access an online social network, in contrast to a conventional personal digital assistant (PDA).

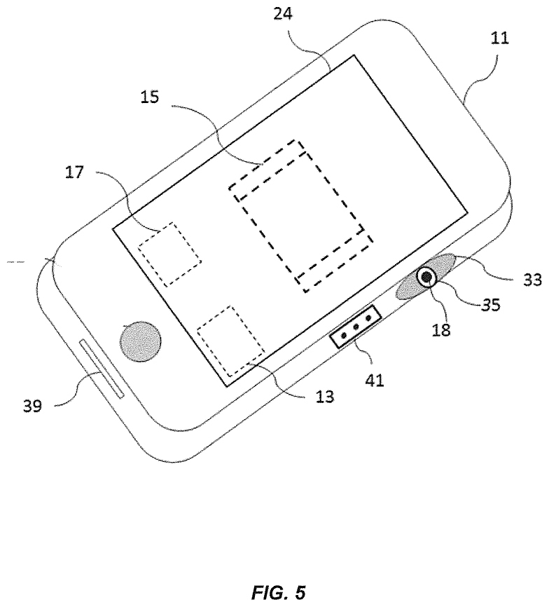

Referring to FIG. 5, an embodiment of EMD 10 is shown based on a web-enabled, mobile phone 11 with biosensors integrated into the casing of the phone. The phone incorporates one or more biosensors 18 to measure physiological parameters that relate to changes in emotional states, such as skin conductance, skin temperature, heart rate, blood volume pulse, blood oxygenation, and electrocardiogram. For a variety of these signals, either wet or dry electrodes, or optical sensors, are utilized. The biosensors may be located in a depression 33 to facilitate finger contact. Alternatively, there may be an array of biosensors, conductive strip, optical fibers, or other means 41 to enable a subject's fingers to be in different positions but still connect to the biosensors, and which allows those biosensors with the strongest signal source to be selected and others used for artifact detection or noise cancellation. A pressure or touch-sensitive sensor 35 in juxtaposition to the biosensors measures finger contact to assist in the detection of artifact. The biosensors are connected to a SPU 17 which amplifies and processes the physiological signals to remove artifact using techniques described above. An accelerometer 13 may be included to aid monitoring and cancellation of movement artifacts.

An application program 15 is downloaded to the mobile phone to derive and display emotion data on screen 24 as previously described. The emotion-deriving algorithms may be implemented in firmware in the mobile phone, in which case the application program receives and displays the emotion data. The emotion data may be integrated with other features of the application, such as a game or personal training program. The emotion data optionally may be transmitted to an internet server, and the emotion data of other users displayed as described above. It will be clear to one of ordinary skill in the art given this teaching that biosensors may similarly be integrated into other types of handheld devices in place of mobile phone, such as a tablet, laptop computer, PC, game controller, TV remote controller, computer mouse, toy, or wearable device, such as a smart watch.

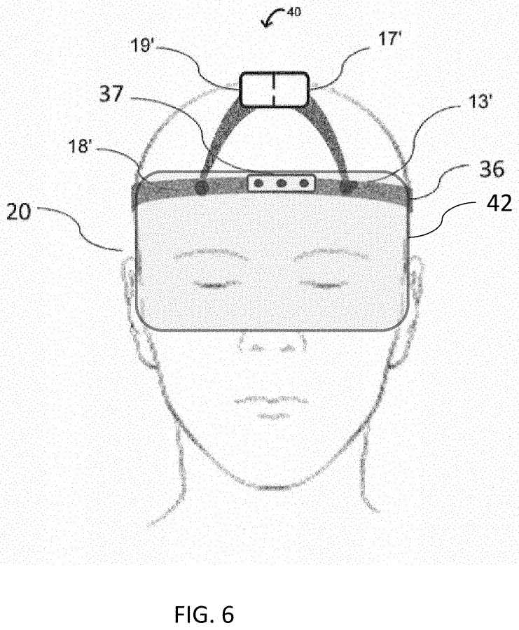

Referring to FIG. 6, an embodiment of EMD 10 is shown based on a wearable headset 40 and virtual reality headset 42. The headset incorporates one or more biosensors 18′ to measure physiological parameters that relate to changes in emotional states, such as EEG, skin conductance, skin temperature, heart rate variability, blood pulse, blood oxygenation, EMG, or ECG. For a variety of these signals, either wet or dry electrodes, or optical sensors, are utilized. Alternatively, there may be an array of biosensors, conductive strips, optical fibers or other means 37 which allow those biosensors with the strongest signal source to be selected and others used for artifact detection or noise cancellation. The biosensors are connected to a SPU 17′ which amplifies and processes the physiological signals to remove artifact using techniques described above. An accelerometer 13′ may be included to aid monitoring and cancellation of movement artifacts.

SPU 17′ may connect with a mobile phone by means of a short-range wireless transmitter 19′ (e.g., Bluetooth®), as illustrated in FIG. 4. The physiological data is transmitted to the phone and an application program running on the mobile phone derives emotion data. Alternatively, the emotion data may be derived by SPU 17′ and transmitted to the mobile phone. The SPU may also transmit the emotion data to device 36 that depicts the current emotional state of the user 20. The emotion data can be displayed, transmitted to the internet, stored on a server, and shared with other users, as described above. The short-range wireless transmitter 19′ may be replaced by a cellular or other telecommunications means that connects directly with the internet. In some cases, the headset may be integrated with a virtual reality display.

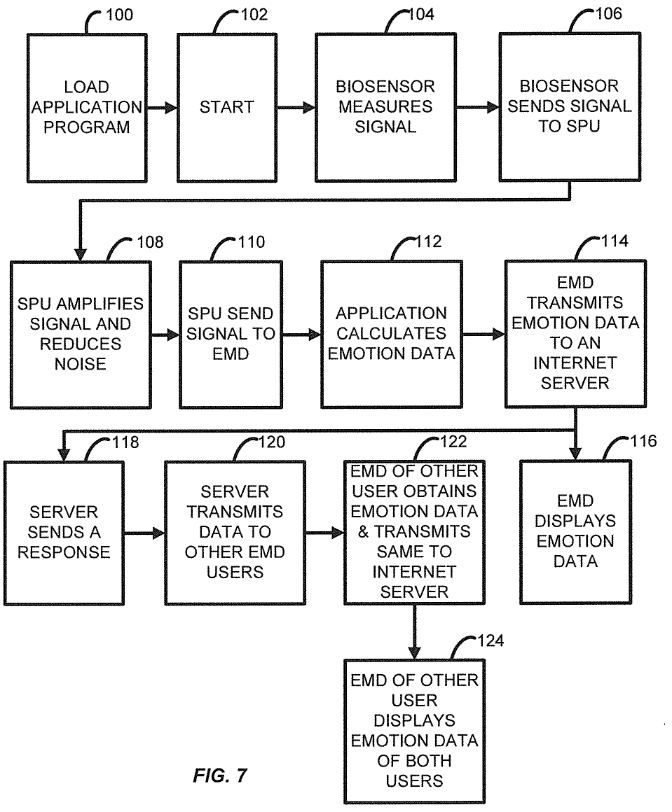

Referring to FIG. 7, a flowchart for a method of operating an emotion monitoring network is illustrated. A user starts an application program (which in some implementations may constitute a very thin client, while in others may be very substantial) in an EMD (step 102), the application program having been previously loaded into the EMD (step 100). A biosensor measures a physiological signal (step 104). The biosensor sends the signal to a SPU (step 106) which amplifies the signal and reduces artifact and noise in the signal (step 108). The SPU transmits the processed signal via a wired or wireless connection to the EMD (step 110). The EMD further processes the signal and calculates a variety of emotion related data, such as emotional arousal and valence measures (step 112). The EMD displays the emotion data to the user (step 116) and transmits the emotion data to an internet server via a telecommunications network (step 114). An application program resident on the internet server processes the emotion data and sends a response to the user (step 118). It should be noted that the application program may reside on one or more servers or cloud infrastructure connected to the internet and the term “response” here is used generally.

Depending on implementation, the internet server may then transmit the emotion data to one or more remote users equipped with an EMD (step 120) where the emotion data are displayed (step 124). The remote user's EMD similarly calculates their emotion data from physiological signals and transmits it to an internet server to be shared with other users (step 122). The sharing may be accomplished in a number of ways, and for a number of purposes. In some cases, aggregate emotional data may be combined and analyzed statistically according to the requirements of the user or user. In other cases, individual emotional data may be employed to notify another user or a group of users of an individual or subject user's emotional state. In still other cases, individual emotional data may be employed to control an avatar in a multiplayer game. In general, a signal corresponding to emotional data may be employed as the basis for calculation, where the calculation is in a videogame, social community, control system, dating or matchmaking network, or the like.

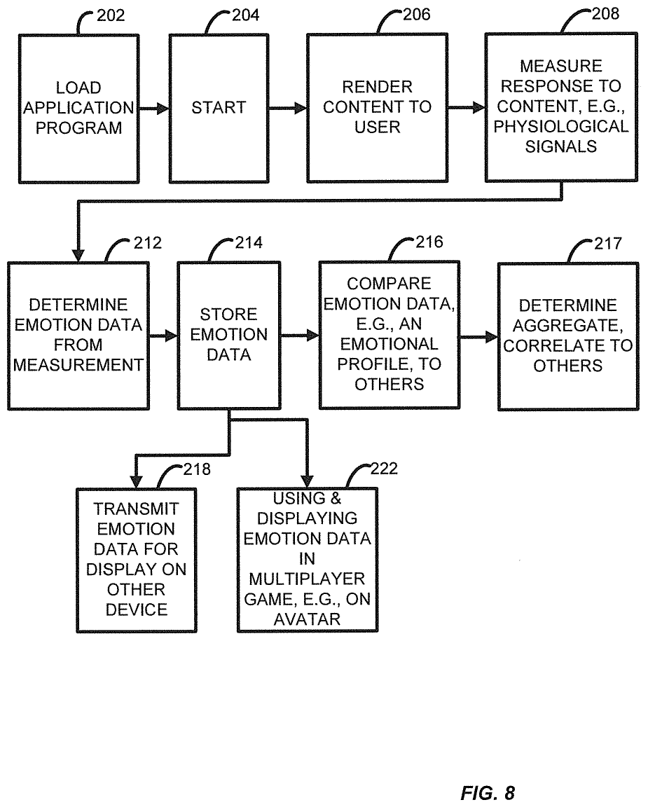

In an implementation for online dating, and referring to FIG. 8, a first step in a method according to present principles is to load an application program (step 202), and start the application (step 204). Content is then rendered to one or more users (step 206). The content may be a series of images, videos, and/or audio items, or a combination of these. A user's response to the content is then measured (step 208), generally resulting in a series of physiological signals from a biosensor. The user's response may be measured by the techniques described above. Emotion data is then determined from the measurement (step 212). The emotion data may include, e.g., emotion arousal and valence components or indices, or other such emotion data. The same may then be stored (step 214). In some implementations the emotion data may be embodied by an emotional profile, which may be a standardized set of indices corresponding to a user's responses to various standardized stimuli.

A variety of other steps may then be taken depending on implementation. For example, emotion data may be compared to others, either individually or within an aggregate (step 216), such as for a dating service. Such a step may be performed to determine an overall emotional character of a group. Emotion data may be transmitted for display on another device (step 218). This type of step is applicable to embodiments such as illustrated in FIG. 2. In another implementation, the data is employed in a multiplayer dating game (step 222). Physiological data may be received from a number of players in the multiplayer game. An emotional profile may be determined from the received physiological data, and an indicator of the emotional profile may be rendered for each player. In this way, the emotional response of a number of users may be displayed, either as a result of interactions or as a result of response to stimuli.

For a dating service such as an online dating service, the data is compared and correlated (step 217). For example, that of a first user may be correlated with those of a plurality of other users, and the user may be presented with a subset of the plurality, i.e., those having correlations greater than a predetermined threshold. The correlation be based on a number of factors, e.g., a emotional compatibility. The emotional compatibility may be based on an external metric, e.g., of the success of dating or long-term relationships.

In another implementation, the comparison may simply be a report of stored indicators of the physiological signals from one, two, or more users. In some cases, an indicator may also be provided of the compatibility of the users, based on the stored indicators.

It will be understood that the above description of the apparatus and method has been with respect to particular embodiments of the invention. While this description is fully capable of attaining the objects of the invention, it is understood that the same is merely representative of the broad scope of the invention envisioned, and that numerous variations of the above embodiments may be known or may become known or are obvious or may become obvious to one of ordinary skill in the art, and these variations are fully within the broad scope of the invention. For example, while certain wireless technologies have been described herein, other such wireless technologies may also be employed. In another variation that may be employed in some implementations of the invention, the measured emotion data may be cleaned of any metadata that may identify the source. Such cleaning may occur at the level of the mobile device or at the level of the secure server receiving the measured data. In addition, it should be noted that while implementations of the invention have been described with respect to sharing emotion data over the internet, e.g., for online dating, multiplayer gaming, or social networking purposes, the invention also encompasses systems in which no such sharing is performed. For example, a user may simply wish to receive a quantitative display of measurements corresponding to their own or another's emotional response over a time period or to a specific stimulus. Accordingly, the scope of the invention is to be limited only by the claims appended hereto, and equivalents thereof. In these claims, a reference to an element in the singular is not intended to mean “one and only one” unless explicitly stated. Rather, the same is intended to mean “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present invention is intended to be dedicated to the public regardless of whether the element, component, or method step is explicitly recited in the claims.

1. A method for capturing, monitoring, and sharing emotion data of two participants in a virtual dating interaction, comprising:
establishing signal communications over a telecommunications network between a server and a first user computing device having a first virtual or augmented reality display, the server operating a server application providing the virtual dating interaction, the first user computing device coupled to one or more first sensors, the one or more first sensors measuring first physiological signals, the first physiological signals being received at the first user computing device;
establishing signal communications over a telecommunications network between the server and a second user computing device having a second virtual or augmented reality display, the second user computing device coupled to one or more second sensors, the one or more second sensors measuring second physiological signals, the second physiological signals being received at the second user computing device;
converting the first and second physiological signals to respective first and second emotion data;
displaying an indication of the first emotion data within the second virtual or augmented reality display; and
displaying an indication of the second emotion data in the first virtual or augmented reality display,
such that an objectively-measured indicator of emotion is shared to each participant to increase objective knowledge of a potential partner for a relationship.

2. The method of claim 1, wherein the converting the first and second physiological signals to respective first and second emotion data is performed at the respective first and second user computing devices.

3. The method of claim 1, wherein the converting the first and second physiological signals to respective first and second emotion data is performed by the server application.

4. The method of claim 1, further comprising calculating a compatibility metric based on the first and second emotion data by the server application, the first user computing device, the second user computing device, or by a combination, and displaying an indication of the compatibility metric on the first or second virtual or augmented reality displays, or both.

5. The method of claim 1, wherein the one or more first sensors or the one or more second sensors, or both, are integrated in the respective first or second virtual reality or augmented reality device.

6. The method of claim 1, further comprising capturing and recording signals from each sensor while a series of objective stimuli are rendered on the first and second virtual or augmented reality displays.

7. The method of claim 1, further comprising capturing and recording signals from each sensor while an interactive media experience is being rendered to the participants on the first and second virtual or augmented reality displays.

8. The method of claim 1, further comprising detecting facial expressions or eye movements using one or more cameras in the first or second virtual or augmented reality display or both, and converting the facial expressions to emotion data, and using the emotion data in the display of indications of the first and second emotion data in the second or first virtual or augmented reality displays, respectively.

9. A non-transitory computer-readable medium, comprising instructions for causing a computing environment to perform a method of capturing, monitoring, and sharing emotion data of two participants in a virtual dating interaction, comprising:
establishing signal communications over a telecommunications network between a server and a first user computing device having a first virtual or augmented reality display, the server operating a server application providing the virtual or augmented reality dating interaction, the first user computing device coupled to one or more first sensors, the one or more first sensors measuring first physiological signals, the first physiological signals being received at the first user computing device;
establishing signal communications over a telecommunications network between the server and a second user computing device having a second virtual or augmented reality display, the second user computing device coupled to one or more second sensors, the one or more second sensors measuring second physiological signals, the second physiological signals being received at the second user computing device;
converting the first and second physiological signals to respective first and second emotion data;
displaying an indication of the first emotion data within the second virtual or augmented reality display; and
displaying an indication of the second emotion data in the first virtual or augmented reality display,
such that an objectively-measured indicator of emotion is shared to each participant to increase objective knowledge of a potential partner for a relationship.

10. A system for capturing, monitoring, and sharing emotion data of two participants in a virtual dating interaction, comprising:
a server application resident on a non-transitory computer readable medium on a server, the server having a server processor;
a first software or firmware application downloaded on a first user computing device, the first user computing device having a first processor;
a second software or firmware application downloaded on a second user computing device, the second user computing device having a second processor;
one or more first sensors configured to be in signal communications with the first user computing device;
one or more second sensors configured to be in signal communications with the second user computing device;
the server processor, the first processor, and the second processor, configured to perform a method, comprising:
establishing signal communications over a telecommunications network between the server and the first user computing device having a first virtual or augmented reality display, the server operating the server application providing the virtual dating interaction, the first user computing device coupled to one or more first sensors, the one or more first sensors measuring first physiological signals, the first physiological signals being received at the first user computing device;
establishing signal communications over a telecommunications network between the server and the second user computing device having a second virtual or augmented reality display, the second user computing device coupled to one or more second sensors, the one or more second sensors measuring second physiological signals, the second physiological signals being received at the second user computing device;
converting the first and second physiological signals to respective first and second emotion data;
displaying an indication of the first emotion data within the second virtual or augmented reality display; and
displaying an indication of the second emotion data in the first virtual or augmented reality display,
such that an objectively-measured indicator of emotion is shared to each participant to increase objective knowledge of a potential partner for a relationship.

11. A method for capturing, monitoring, and sharing emotion data of participants in a virtual social interaction, comprising:
establishing signal communications over a telecommunications network between a server and a first user computing device having a first virtual or augmented reality display, the server operating a server application providing a social interaction, the first user computing device coupled to one or more first sensors, the one or more first sensors measuring first physiological signals, the first physiological signals being received at the first user computing device;
establishing signal communications over a telecommunications network between the server operating the server application providing the social interaction and one or more other user computing devices each having a respective other virtual or augmented reality display, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data;
displaying an indication of the first or other emotion data,
whereby social dynamics within the participants in the social interaction is enhanced by determination and use of objective emotion data of the participants.

12. The method of claim 11, wherein the indication is displayed within one of the first or other virtual or augmented reality displays.

13. The method of claim 11, wherein the indication of the first emotion data is displayed within one or more of the other virtual or augmented reality displays, or wherein the indication of the one or more other emotion data is displayed within the first virtual or augmented reality display, or both.

14. The method of claim 11, wherein the converting the first and other physiological signals to respective first and other emotion data is performed at the respective first and other user computing devices.

15. The method of claim 11, wherein the converting the first and other physiological signals to respective first and other emotion data is performed by the server application.

16. The method of claim 11, further comprising calculating an aggregate of the emotion data based on the first and other emotion data by the server application, the first user computing device, the one or more other user computing devices, or by a combination, and displaying an indication of the aggregate emotion data on the first or other virtual or augmented reality displays, or both, or at an external display device in network communication with the server.

17. The method of claim 11, wherein the one or more first sensors or the one or more other sensors, or both, are integrated in the respective first or one or more other virtual or augmented reality displays.

18. The method of claim 11, further comprising capturing and recording signals from the one or more first sensors and the one or more other sensors while an interactive media experience is being rendered to the participants on the first and other virtual or augmented reality displays.

19. The method of claim 18, wherein the interactive experience is a dating experience, game, teambuilding or recruiting exercise, or biofeedback training, and wherein the first and other emotion data are used as an input to the dating experience, game, teambuilding or recruiting exercise, or biofeedback training.

20. The method of claim 11, further comprising correlating at least the first emotion data and one or more of the other emotion data, and displaying an indication of the correlation on the first virtual or augmented reality display, on one or more of the other virtual or augmented reality displays, or on an external display device.

21. The method of claim 11, further comprising detecting facial expressions or eye movements using one or more cameras in one or more of the first or other virtual or augmented reality displays, and converting the facial expressions to emotion data, and using the emotion data in the display of indications of the first or other emotion data.

22. A non-transitory computer-readable medium, comprising instructions for causing a computing environment to perform a method of capturing, monitoring, and sharing emotion data of participants in a virtual social interaction, comprising:
establishing signal communications over a telecommunications network between a server and a first user computing device having a first virtual or augmented reality display, the server operating a server application providing a social interaction, the first user computing device coupled to one or more first sensors, the one or more first sensors measuring first physiological signals, the first physiological signals being received at the first user computing device;
establishing signal communications over a telecommunications network between the server operating the server application providing the social interaction and one or more other user computing devices each having a respective other virtual or augmented reality display, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data;
displaying an indication of the first or other emotion data,
whereby social dynamics within the participants in the virtual social interaction is enhanced by determination and use of objective emotion data of the participants.

23. A system for capturing, monitoring, and sharing emotion data of participants in a virtual social interaction, comprising:
a server application resident on a non-transitory computer readable medium on a server, the server having a server processor;
a first software or firmware application downloaded on a first user computing device, the first user computing device having a first processor;
another software or firmware application downloaded on one or more respective other user computing devices, the respective other computing devices having respective other processors;
one or more first sensors configured to be in signal communications with the first user computing device;
one or more other sensors configured to be in signal communications with each of the one or more other computing devices, respectively;
the server processor, the first processor, and the other processors, configured to perform a method, comprising:
establishing signal communications over a telecommunications network between a server and a first user computing device having a first virtual or augmented reality display, the server operating a server application providing a social interaction, the first user computing device coupled to one or more first sensors, the one or more first sensors measuring first physiological signals, the first physiological signals being received at the first user computing device;
establishing signal communications over a telecommunications network between the server operating the server application providing the social interaction and one or more other user computing devices each having a respective other virtual or augmented reality display, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data;
displaying an indication of the first or other emotion data,
whereby social dynamics within the participants in the virtual social interaction is enhanced by determination and use of objective emotion data of the participants.

24. A method for capturing, monitoring, and sharing emotion data of participants in a video call, comprising:
establishing signal communications over a telecommunications network between a server and a first user computing device having a first video display, the server operating a server application providing a video call, the first user computing device coupled to one or more first sensors, the one or more first sensors measuring first physiological signals, the first physiological signals being received at the first user computing device;
establishing signal communications over a telecommunications network between the server operating the server application providing the social interaction and one or more other user computing devices each having a respective other video display, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data; and
displaying an indication of the first or other emotion data on the first video display or on one or more of the other video displays, or on a combination,
whereby social dynamics within the participants in the video call is enhanced by determination and use of objective emotion data of the participants.

25. A non-transitory computer-readable medium, comprising instructions for causing a computing environment to perform a method of capturing, monitoring, and sharing emotion data of participants in a video call, comprising:
establishing signal communications over a telecommunications network between a server and a first user computing device having a first video display, the server operating a server application providing a video call, the first user computing device coupled to one or more first sensors, the one or more first sensors measuring first physiological signals, the first physiological signals being received at the first user computing device;
establishing signal communications over a telecommunications network between the server operating the server application providing the social interaction and one or more other user computing devices each having a respective other video display, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data; and
displaying an indication of the first or other emotion data on the first video display or on one or more of the other video displays, or on a combination,
whereby social dynamics within the participants in the video call is enhanced by determination and use of objective emotion data of the participants.

26. A system for capturing, monitoring, and sharing emotion data of participants in a video call, comprising:
a server application resident on a non-transitory computer readable medium on a server, the server having a server processor;
a first software or firmware application downloaded on a first user computing device, the first user computing device having a first processor;
another software or firmware application downloaded on one or more respective other user computing devices, the respective other computing devices having respective other processors;
one or more first sensors configured to be in signal communications with the first user computing device;
one or more other sensors configured to be in signal communications with each of the one or more other computing devices, respectively;
establishing signal communications over a telecommunications network between a server and a first user computing device having a first video display, the server operating a server application providing a video call, the first user computing device coupled to one or more first sensors, the one or more first sensors measuring first physiological signals, the first physiological signals being received at the first user computing device;
establishing signal communications over a telecommunications network between the server operating the server application providing the social interaction and one or more other user computing devices each having a respective other video display, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data; and
displaying an indication of the first or other emotion data on the first video display or on one or more of the other video displays, or on a combination,
whereby social dynamics within the participants in the video call is enhanced by determination and use of objective emotion data of the participants.

27. A method for capturing, monitoring, and sharing emotion data of participants in a virtual business conference, comprising:
establishing signal communications over a telecommunications network between a server and a first user computing device having a first virtual or augmented reality display, the server operating a server application providing a virtual business conference, the first user computing device coupled to one or more first sensors, the one or more first sensors measuring first physiological signals, the first physiological signals being received at the first user computing device;
establishing signal communications over a telecommunications network between the server operating the server application providing the virtual business conference and one or more other user computing devices each having respective other virtual or augmented reality displays, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data;
displaying an indication of the first or other emotion data,
whereby social dynamics within the participants in the virtual business conference is enhanced by determination and use of objective emotion data of the participants.

28. The method of claim 27, wherein the indication is displayed within one of the first or other virtual or augmented reality displays.

29. The method of claim 27, wherein the indication of the first emotion data is displayed within one or more of the other virtual or augmented reality displays, or wherein the indication of the one or more other emotion data is displayed within the first virtual or augmented reality display, or both.

30. The method of claim 27, wherein the converting the first and other physiological signals to respective first and other emotion data is performed at the respective first and other user computing devices.

31. The method of claim 27, wherein the converting the first and other physiological signals to respective first and other emotion data is performed by the server application.

32. The method of claim 27, further comprising calculating an aggregate of the emotion data based on the first and other emotion data by the server application, the first user computing device, the one or more other user computing devices, or by a combination, and displaying an indication of the aggregate emotion data on the first or other virtual or augmented reality displays, or both, or at an external display device in network communication with the server.

33. The method of claim 27, wherein the one or more first sensors or the one or more other sensors, or both, are integrated in the respective first or one or more other virtual or augmented reality devices.

34. The method of claim 27, further comprising capturing and recording signals from the one or more first sensors and the one or more other sensors while an interactive media experience is being rendered to the participants on the first and other virtual or augmented reality displays.

35. The method of claim 27, further comprising correlating at least the first emotion data and one or more of the other emotion data, and displaying an indication of the correlation on the first virtual or augmented reality display, on one or more of the other virtual or augmented reality displays, or on an external display device.

36. The method of claim 27, further comprising detecting facial expressions or eye movements using one or more cameras in one or more of the first or other virtual or augmented reality displays, and converting the facial expressions to emotion data, and using the emotion data in the display of indications of the first or other emotion data.

37. A non-transitory computer-readable medium, comprising instructions for causing a computing environment to perform a method of capturing, monitoring, and sharing emotion data of participants in a virtual business conference, comprising:
establishing signal communications over a telecommunications network between a server and a first user computing device having a first virtual or augmented reality display, the server operating a server application providing a virtual business conference, the first user computing device coupled to one or more first sensors, the one or more first sensors measuring first physiological signals, the first physiological signals being received at the first user computing device;
establishing signal communications over a telecommunications network between the server operating the server application providing the virtual business conference and one or more other user computing devices each having respective other virtual or augmented reality displays, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data;
displaying an indication of the first or other emotion data,
whereby social dynamics within the participants in the virtual business conference is enhanced by determination and use of objective emotion data of the participants.

38. A system for capturing, monitoring, and sharing emotion data of participants in a virtual business conference, comprising:
a server application resident on a non-transitory computer readable medium on a server, the server having a server processor;
a first software or firmware application downloaded on a first user computing device, the first user computing device having a first processor;
another software or firmware application downloaded on one or more respective other user computing devices, the respective other computing devices having respective other processors;
one or more first sensors configured to be in signal communications with the first user computing device;
one or more other sensors configured to be in signal communications with each of the one or more other computing devices, respectively;
the server processor, the first processor, and the other processors, configured to perform a method, comprising:
establishing signal communications over a telecommunications network between a server and a first user computing device having a first virtual or augmented reality display, the server operating a server application providing a virtual business conference, the first user computing device coupled to one or more first sensors, the one or more first sensors measuring first physiological signals, the first physiological signals being received at the first user computing device;
establishing signal communications over a telecommunications network between the server operating the server application providing the virtual business conference and one or more other user computing devices each having respective other virtual or augmented reality displays, the one or more other user computing devices coupled to respective one or more other sensors, the respective one or more other sensors measuring one or more other physiological signals, the one or more other physiological signals being received at the one or more other user computing devices, respectively;
converting the first and other physiological signals to respective first and other emotion data;
displaying an indication of the first or other emotion data,
whereby social dynamics within the participants in the virtual business conference is enhanced by determination and use of objective emotion data of the participants.
